(19) United States
(12) Patent Application Publication
(10) Pub. No.: US 2007/0249030 A1
(43) Pub. Date: Oct. 25, 2007

(54) Methods, Apparatus, Products and Compositions Useful for Processing Fermentation Waste Streams

(76) Inventors: Michael Charles Fahrenthold, Bastrop, TX (US); Paul David Fahrenthold, La Grange, TX (US)

Correspondence Address: GILBRETH & ASSOCIATES, P.C., PO BOX 2428, BELLAIRE, TX, 77402-2428, US

(21) Appl. No.: 11/739,081

(22) Filed: Apr. 23, 2007

Related U.S. Application Data
(60) Provisional application No. 60/767,498, filed on Apr. 23, 2006.
(60) Provisional application No. 60/767,504, filed on Apr. 26, 2006.

(51) Int. Cl. C12P 7/06

(52) U.S. Cl. 435/161

(57) ABSTRACT

A method for processing a stream from a fermentation process, wherein the stream comprises polysaccharides, the method comprising, contacting the stream with an enzyme to convert at least a portion of the polysaccharides into a fermentable sugar to form a fermentable sugar stream.

### RELATED APPLICATION DATA

The present application claims priority of U.S. Provisional Patent Application 60/767,498, filed Apr. 23, 2006, and U.S. Provisional Patent Application 60/767,504, filed Apr. 26, 2006, the specifications of which are both herein incorporated by reference.

### BACKGROUND OF THE DISCLOSURE

1. Field of the Disclosure

The present disclosure relates to methods, apparatus, compositions and products for conversion of polysaccharides into sugars. In another aspect, the present disclosure relates to methods, apparatus, compositions, and products for conversion of polysaccharides in fermentation waste streams into fermentable sugars. In even another aspect, the present disclosure relates to methods, apparatus, compositions, and products for processing waste streams from fermentation processes. In still another aspect, the present disclosure relates to methods, apparatus, compositions, and products for conversion of polysaccharides in fermentation waste streams from molasses based fermentation, into fermentable sugars. In yet another aspect, the present disclosure relates to methods, apparatus, compositions, and products for conversion of polysaccharides in fermentation waste streams from rum production, into fermentable sugars. In even still another aspect, the present disclosure relates to methods, apparatus, compositions, and products for conversion of polysaccharides in fermentation waste streams from fuel or industrial grade ethanol production, into fermentable sugars. In even yet another aspect, the present disclosure relates to methods, apparatus, compositions, and products for fermentation.

2. Description of the Related Art

In a typical fermentation process for making alcohol, a yeast is contacted with an aqueous sugar solution to make a fermenter broth, which after a certain period of time will ferment into a solution comprising ethanol.

Of course, the sugar solutions will vary with the type of alcohol desired. For example, in the making of rum, yeast is mixed with molasses, and in about 48 hours, the broth has fermented to about 10% by volume ethanol.

This fermented broth is now sent to a distillation unit in which low boiling organics and the ethanol is distilled off, and a bottom waste stream formed.

In the rum making process, this broth, now called “beer”, is sent to a beer still to remove the low boiling organics and all of the alcohol. The beer still generates a waste from the bottom called “mostos”, “vinasse” or “dunder”. This waste stream will comprise among other things, polysaccharides, and various organic acids such as lactic acid, acetic acid, formic acid, and proprionic acid.

Very commonly, this waste stream is dumped or discarded.

Unfortunately, this waste stream contains materials that could have a negative impact on the environment.

Thus, it would be desirable to process part or all of this waste stream into a useful stream, to recover useful materials from this waste stream, or to convert some or all of this stream into useful materials.

### BRIEF DESCRIPTION OF THE DRAWINGS

FIG. 1 shows a schematic representation of some optional pretreatment steps, including, dissolved air filtering, filtering, and implementation of nano-filter technology to remove some organics.

FIG. 2 shows a flowchart of one non-limiting embodiment of the isolation and conversion of polysaccharides to alcohol.

FIG. 3 is a schematic representation of some optional treatment steps, including acid addition, heating to the reaction temperature in a pressure vessel, cooling the acid treated material and neutralization prior to enzyme treatment.

### SUMMARY OF THE DISCLOSURE

According to one embodiment, there is provided, a method for processing a stream from a fermentation process, wherein the stream comprises polysaccharides, the method comprising, contacting the stream with an enzyme to convert at least a portion of the polysaccharides into a fermentable sugar to form a fermentable sugar stream.

According to another embodiment, there is provided a fermentation method comprising: A. Contacting a fermentable sugar and yeast in a fermenter broth to form ethanol and a polysaccharide; B. Recovering at least a portion of the polysaccharide as a recovered polysaccharide; and C. Contacting the recovered polysaccharide with an enzyme.

According to even another embodiment of the present invention, there is provided a product comprising a stream from a fermentation process comprising polysaccharides, and an enzyme.

According to still another embodiment, there is provided a method for processing a stream from a fermentation process, wherein the stream comprises polysaccharides and organic acids, the method comprising, separating the stream into a concentrated polysaccharide portion having a higher polysaccharide concentration than the stream, and an organic acids portion having a higher acids concentration than the stream.

### DETAILED DESCRIPTION OF THE DISCLOSURE

The present disclosure has application to fermentation processes that produce streams comprising polysaccharides. These polysaccharide containing streams may be processed by contacting them with at least one enzyme to convert the polysaccharide to another polysaccharide, oligosaccharide, and/or fermentable sugar.

Such fermentation processes include but are not limited to yeast manufacture from molasses, pharmaceutical manufacture by fermentation, ethanol from corn, ethanol from fermentation of cane juice, ethanol from fermentation of any sugar source, sugar production, etc.

The polysaccharide stream, may comprise one or more types of polysaccharides, depending upon the process from which it was obtained. As a non-limiting example, from one type of rum making processes, the polysaccharide will comprise mainly glucose and galactitol, in addition to smaller amounts of mannose, and trace amounts of xylose, arabinose, rhamnose and galactose.

The polysaccharide-containing stream will comprise a wide weight percent of polysaccharides depending upon the process from which it is obtained. It should also be understood that the polysaccharide stream may be processed to concentrate the weight percentage of polysaccharide prior to contact with the enzyme.

For example, the polysaccharide stream may comprise in the range of about 0.01 to about 100 percent by weight of the polysaccharide, but most likely if obtained directly from a fermentation process will comprise in the range of about 0.01 to about 25 weight percent polysaccharide, even more likely, in the range of about 2 to about 20 weight percent polysaccharide, and still more likely, in the range of about 4 to about 12 weight percent polysaccharide. As a specific non-limiting example, a waste stream from certain rum making processes contain about 8-10 weight percent polysaccharide.

Prior to any enzyme treatment, a number of optional pretreatment steps may be utilized to make the polysaccharide stream more susceptible to enzyme treatment. As a non-limiting example, it is sometimes desirable to concentrate the polysaccharide stream to increase the percentage of polysaccharide in the stream. Thus, optionally, any number of concentrating techniques could be utilized to concentrate the polysaccharides.

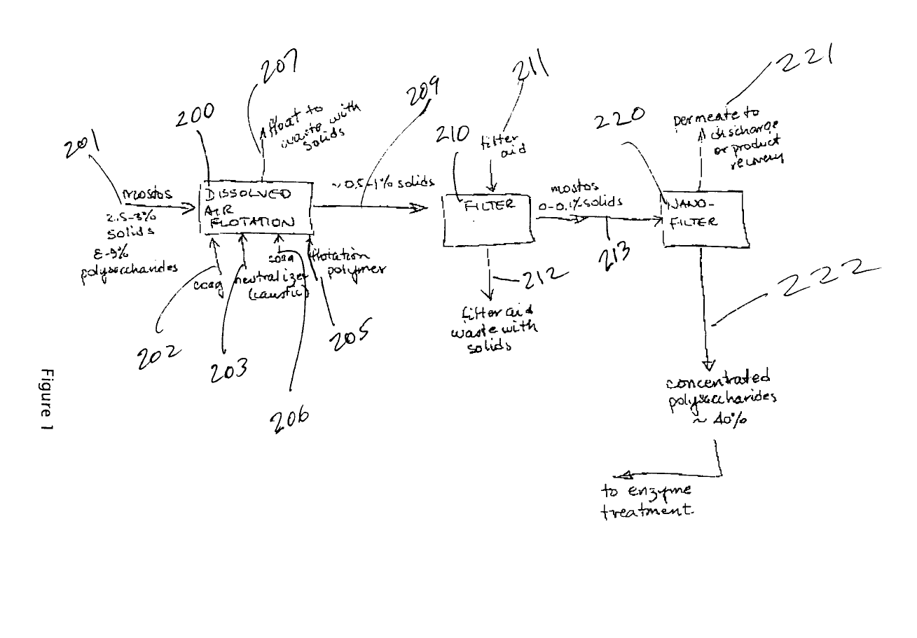

As a non-limiting example, FIG. 1 shows a schematic representation of some optional pretreatment steps, including, dissolved air flotation 200, filtering 210, and nano-filter technology 220 to remove some organics.

As contemplated in FIG. 1, the polysaccharide stream 201 may optionally be processed by DAF (Dissolved Air Flotation), which separates solids from liquids through the use of a polymer that attaches to the solids and to small air bubbles.

As a non-limiting example, polysaccharide stream 201, which may be mostos from a rum process containing 2.5-3% solids, 8-9% polysaccharides, and having a pH of 4.2-4.7, is transferred from the bottom of the beer still to DAF device 200. A series of metering pumps adds processing chemicals, coagulant 202 (additional coagulant 206 may also be added), neutralizer 203, and floatation polymer 205, to the mostos to insure that the solids in the mostos are coagulated, neutralized and floated to the surface of the mostos where the solids can be readily skimmed from the surface (stream 207).

It should be understood that any number of suitable processing chemicals may optionally be added to the polysaccharide stream, non-limiting examples of which include the following.

Aluminum chloride: this chemical acts as a coagulant. It is added at a concentration of approximately 40 ppm in mostos.

Sodium hydroxide: this chemical is added to mostos to neutralize the acids formed in the fermentation of molasses so that the polysaccharide stream will have a pH suitable to allow operating of the DAF, typically in the range of about 6.8-7.3, but may be lower or higher depending upon the coagulant and polymer utilized. Specifically, without a suitable pH, the added polymers may not function adequately to further coagulate the solids in mostos and float them to the surface of the mostos where they can be removed from the DAF device.

Coagulant: this chemical is an organic polymer that coagulates the solids attached to the aluminum chloride. As stated above, the pH is typically in the range of about 6.8-7.3. A commercially available polymer suitable to accomplish this coagulation is from Polymer Research Corporation, having the product designation 3070BX. It is generally added to mostos adequate to provide a concentration of 1-5 ppm in the mostos.

Flotation Polymer: This polymer is added to the mostos in order to facilitiate the removal of solids by attaching the coagulated solids to small air bubbles so that the solids can be removed from the system. A commercially available polymer suitable to accomplish this action is from Kan-Floc Corp., identified as Kan-Floc 4500m. It is generally added to mostos adequate to provide a concentration of 1-5 ppm in the mostos.

As a non-limiting example, for polysaccharide streams from certain rum processes, the pH of the incoming mostos is raised from 4.2-4.5 (stream 201) up to 6.8-7.3 (stream 209), and the solids content is reduced from 2.5-3% (steam 201) down to ˜0.5-1% (stream 209).

As further illustrated in FIG. 1, filtration 210 may be utilized to take the solids content lower. Specifically, in the case of mostos filtration any of the common filtration devices would be suitable: plate and frame, rotary vacuum, horizontal tank-vertical leaf, etc.

As a non-limiting example, any filtering may be enhanced with the use of a filter aid 212, a suitable commercially available product is Eagle Picher type FW-60. Through the use of filtering, more of the residual solids are removed through stream 212. The entering solids content of ˜0.5-1% solids is reduced to the range of 0.0-0.1% (stream 213).

Referring still to FIG. 1, nanofilter 220, a device that removes low molecular weight compounds in solution from higher molecular weight compounds in solution, is utilized to remove water and organic acids.

In the case of mostos nanofiltration the objective of the process is to remove some or all of the water and low molecular weight organic compounds from mostos (in a stream 222 called the “permeate”), producing an aqueous stream 222 containing higher concentrations of polysaccharides (high molecular weight organics) in the rejected (called the “reject”) stream 222 than in the inlet mostos stream 213 from the filter.

Passage of filtered mostos through the nanofilter results in the concentration of polysaccharides in mostos. Normal concentration ratios are from 2-5, although more concentrations can be possible depending upon the number of times the mostos is passed through the filter (either repeated passes through a few filters or one pass through many filters). In a typical application a concentration ratio of approximately 5 would be used to produce a mostos with a polysaccharide concentration of about 40% (stream 222).

The permeate stream 221 can contain common acids such as lactic, formic, acetic, propionic, etc. some simple sugars such as glucose, fructose, sucrose, and water. Of course the composition of the permeate stream will vary depending upon the origin of the polysaccharide stream. For example, for streams obtained from a rum process, it would not be uncommon for the permeate stream to have a concentration of lactic acid, the acid in the largest concentration, in the range of 3,000-7,000 ppm, with the other organic acids and the sugars, present at relative low levels on the order of a few hundred ppm or less.

This disclosure contemplates a process for the recovery from stream 222 of these organic acids, as well as products comprising those organic acids.

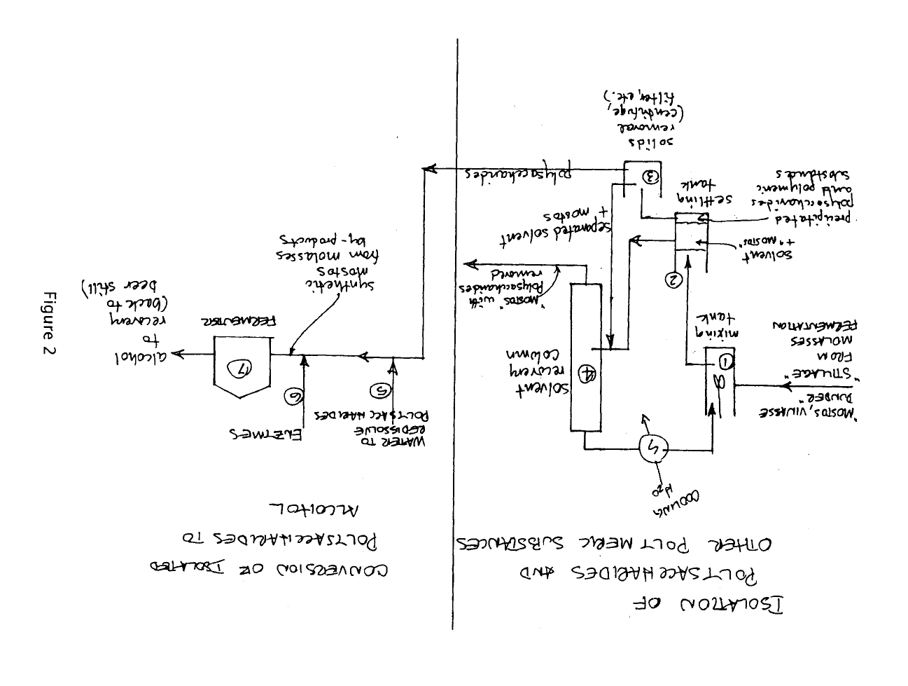

Referring now to FIG. 2, there is shown a flow diagram of another embodiment for the pretreatment of stillage (“mostos”, “vinasse” or “dunder”), for example from the distillation of fermented molasses. In this embodiment, stillage may be pretreated by mixing the stillage in mixing tank 1 with a water miscible organic solvent to concentrate the polysaccharides contained therein.

While any suitable water miscible organic solvent may be utilized, ethanol may conveniently be utilized as it is commonly available at distilleries. Another non-limiting example of a suitable solvent is acetone. The solvent is added in any suitable amount. As a non-limiting example, the solvent may generally be added in a quantity of at least one volume of solvent for each volume of mostos, and may preferably be added in a quantity of at least two volumes of solvent per volume of mostos. Some solvents, a non-limiting example of which includes ethanol, may contain an azeotropic volume of water and still be effective in the process.

The mixture may be mixed by any suitable method utilizing any suitable means, for any suitable mixing time. As a non-limiting example, the mixture may be mixed mechanically for a short time, perhaps having a residence time in the mixing vessel as long as 1-5 minutes.

In some instances, the solids may be present in the mostos and solvent mixture. Those solids may be removed by any suitable technique. As a non-limiting example, the solids are concentrated in a solution that is then settled in a settling vessel 2. The residence time in the vessel need not be long, perhaps 1-30 minutes. At the end of the settling period the solids will have settled to the bottom of the vessel and the supernatant and solids can be readily separated. The supernatant (solution of solvent and mostos) will be clear of solids and sent to solvent recovery column 4. The settled solids may contain a small quantity of the supernatant and will be sent to solids removal station 3.

The settled solids, consisting of polysaccharides and other polymeric material not soluble in the mostos-solvent mixture, can be further processed at solids removal station 3 in order to produce a solid material and a clear solution that may be equivalent to the above described supernatant which is also sent to solvent recovery column 4. The solids can be separated by any suitable technique, non-limiting examples of which include filtration, centrifugation or other means.

All of the supernatant streams, the clear material from settling tank 2 and that removed from the solids in station 3, may be recovered for recycle back to mixing tank 1. A stripping column 4 can be used for this purpose with the appropriate auxiliary distillation equipment such as heat exchangers and condensers. Mostos, free of the polysaccharides and other polymeric materials (proteins, etc.) may be removed from the bottom high boiling stream from the column. This stream can be used for further recovery of low molecular weight compounds such as lactic acid or low molecular weight polymers. Residual material from recovery of those materials would be suitable for waste treatment by any of a number of processes.

Solids comprised of polysaccharides and other polymeric materials are dissolved in water at station 5 to establish a solids concentration adequate for enzymatic treatment station 6. A solids concentration will generally be in the range of about 1-80 weight percent, preferably in the range of about 5-75 weight percent, more preferably in the range of about 10-50 weight percent, and even more preferably in the range of about 25-35. A solids concentration of 30% may be utilized.

The solution of polysaccharides and other polymeric substances is contacted with enzymes at station 6 to convert the polysaccharides to glucose and other fermentable sugars. Any suitable enzymes may be utilized, including puiluinase, alpha-glucosidase, and alpha-amylase, and any other enzymes previously identified can also be used as suitable for the origin of the molasses that produced the mostos raw material.

The enzyme treated material can be fermented at fermenter 7 with any of a number of yeasts to produce ethanol. The ethanol-water mixture can be separated from the yeast by any suitable technique, a non-limiting example of which includes use of a centrifuge with the yeast recycled to a subsequent fermentation. The ethanol/water solution is then subjected to distillation for recovery of the ethanol produced during fermentation.

Another non-limiting embodiment for pretreatment of the lignocellulosic and polysaccharide components of mostos is hydrolysis using elevated temperatures under acidic conditions.

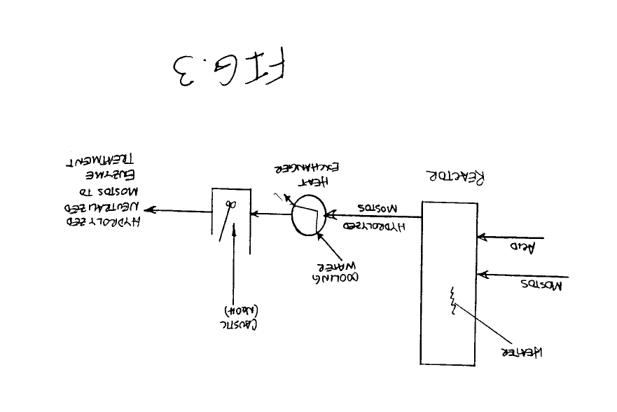

As a non-limiting example, FIG. 3 provides a schematic representation of some optional treatment steps, including acid addition, heating to the reaction temperature in a pressure vessel, cooling the acid treated material and neutralization prior to enzyme treatment.

As contemplated in FIG. 3, the acid added to mostos may be any suitable acid. As a non-limiting example, a suitable acid includes a mineral acid such as sulfuric, hydrochloric, etc. in a concentration from 0.1% to 50% most likely in a range of 0.5% to 5% by weight of the mostos sent to the reactor vessel.

As further contemplated in FIG. 3, the mixture of acid and mostos may be heated for the reaction to occur. Reaction temperatures may range from 150° C. to 250° C., preferably in the range of 180° C. to 225° C.

The mixture of mostos and acid must be held at elevated temperature as specified above to implement the hydrolysis process. Residence times in the reactor can vary from 1 minute to 60 minutes, with a preferred residence time of 1 to 30 minutes.

After a suitable time in the reactor, the reaction mix may be cooled and neutralized with any base material suitable for enzyme treatment. A non-limiting example of a suitable base includes sodium hydroxide.

The concentrated polysaccharides are now concentrated for enzyme treatment for conversion of the polysaccharides to simple sugars. Most commonly, it will be desirable to obtain glucose, fructose, sucrose, and any mixtures thereof.

It should be understood that any enzyme that will convert the particular polysaccharide in the polysaccharide stream to another polysaccharide, an oligosaccharide, and/or fermentable sugar, may be utilized.

Non-limiting examples of suitable enzymes include dextranase, cellulase, galactomannase, hemicellulase, xylanase, fungal amylase, glucoamylase, invertase, bacterial protease, fungal protease, and any combination thereof.

It should be understood that any combination of enzymes may be utilized, whether the combination is added all at once, or sequentially. For example, at various times in the enzyme contacting process, an enzyme or combination of enzymes may be added.

Non-limiting examples of suitable combinations of enzymes include:
fungal amylase and glucoamylase;
dextranase and invertase;
dextranase and cellulase;
dextranase, cellulase and glucoamylase;
dextranase, cellulase, fungal amylase and glucoamylase;
dextranase, cellulase, fungal amylase, glucoamylase, and invertase;
hemicellulase, fungal amylase, glucoamylase;
cellulase, fungal amylase, glucoamylase;
bacterial protease, fungal amylase, glucoamylase;
cellulase, bacterial protease, fungal amylase, glucoamylase;
hemicellulase, bacterial protease, fungal amylase, glucoamylase;
fungal protease, fungal amylase, glucoamylase;
cellulase, fungal protease, fungal amylase, glucoamylase;
hemicellulase, fungal protease, fungal amylase, glucoamylase;
galactomannanase, bacterial protease, fungal amylase, glucoamylase; and
galactomannanase, fungal protease, fungal amylase, glucoamylase.

The contacting of the enzyme with the polysaccharide will take place under suitable process conditions to allow the conversion of the polysaccharide as desired. Generally, the process conditions of interest include one or more of the particular enzyme, particular polysaccharide, contacting time, mixing efficiency, flow rate, concentrations of the enzyme and polysaccharide, solids concentration (i.e., dissolved polysaccharide in solution), temperature, pH, phase conditions, and other process conditions.

The contacting of the polysaccharide with the enzyme may take place in any suitable type of reactor, and may be a batch process or a continuous process as desired. It should be understood that a number of reactions may be utilized, either in parallel, or in series, or any combination thereof. In such systems, both batch and continuous reactors may be utilized.

Multiple reaction zones may be provided by a single reactor having more than one reaction zone, or by a number of reactors each having one or more reaction zones. Variously placed feed inlets for both polysaccharide and enzyme streams may be utilized, along with variously placed outlet product streams.

The enzymes and the polysaccharide stream may each be added to the reactor in any desirable manner, non-limiting examples of which include in one shot, over a period of time at regular or irregular time intervals in equal or unequal dosages, or continuously in equal or unequal rates at a particular time. Furthermore, the contacting of the enzymes and the polysaccharide stream may optionally take place before entering the reactor.

It should be noted, that the reactivity of the enzyme with the polysaccharide may also vary with the ratio of the enzyme to the polysaccharide. Thus, in addition to possibly adding enzymes at various times in the contacting process, it may be necessary to add polysaccharides at various times in the contacting process.

The contacting time is generally selected to allow the desired conversion of polysaccharide to occur, generally taking into account economic factors and process conditions. Generally however, the contacting time of the enzyme with the polysaccharide will be in the range from at least about 0.01 minutes, 1 hour, or 2 hours, ranging up to about 3 hours, 24 hours, or 10 days.

The pH is generally selected to allow the desired conversion of polysaccharide to occur, generally taking into account economic factors and process conditions. Generally however, the pH during the contacting of the enzyme and polysaccharide will be in the range from at least about 2, 3, 4, or 7, ranging up to about 5, 8, 10 or, 12, with a non-limiting example of a suitable range being from 3 pH to 8 pH.

The contacting of the enzyme and polysaccharide will generally take place with sufficient mixing to achieve a conversion of the polysaccharide as desired. Suitable mixing may be provided by impellers, stirrers, shaking, inline mixing, sparging, and the like.

The contacting of the enzyme and polysaccharide will generally take place at a contacting temperature sufficient to achieve a conversion of the polysaccharide as desired. At too low of temperature, enzyme actively will greatly decrease and may stop. At too high of temperature, the enzyme may be destroyed. So, generally, the temperature will need to be within the activity temperature for the particular enzyme. Generally however, the contacting temperature of the enzyme with the polysaccharide will be in the range from at least about 10 C, 20 C, or 40 C, ranging up to about 40 C, 60 C, or 10° C., with a non-limiting example of a suitable range being from 40 C to 60 C.

The contacting of the enzyme and polysaccharide will generally take place at a weight ratio of enzyme:polysaccharide sufficient to achieve a conversion of the polysaccharide as desired. At too low of a ratio, the reaction will proceed to slowly and with too low of conversion. At too high of a ratio, there may be economic waste of the enzyme. Generally, contacting of the enzyme and polysaccharide will take place with the weight ratio of enzyme:polysaccharide ranging from about 0.01, 0.5, 1, or 2 at the low end of the range, up to about 1, 5, 10, or 100, at the upper end of the range.

As for phase conditions, the contacting of the enzyme and polysaccharide may take place between an aqueous solution of the polysaccharide and the enzyme, or the enzyme may be in solid form support on an inert support, or contained on or encapsulated in a bead or pellet.

Once the polysaccharide stream has been treated with enzymes to convert the polysaccharides as desired into other polysaccharides, oligosaccharides, and/or fermentable sugars, it may be utilized in any number of processes as desired, including recycle back into the process from which is was originally obtained, or into another fermentation process.

As a non-limiting example, a rum process polysaccharide stream may be enzyme treated and then recycled back into any point of the rum process or most likely combined with the fermenter feed streams, or added directly to the fermenter.

Depending upon the desired use of the enzyme treated stream, the enzyme treatment may be tailored to produce any desired mixture and concentration of fermentable sugars.

The present invention finds applicability to fermentation processes in general, including but not limited to, molasses based fermentation processes, rum fermentation, and to fermentation to make fuel and industrial grade ethanol.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the drawings, examples, and descriptions set forth herein but rather that the claims be construed as encompassing all the features of patentable novelty which reside in the present invention, including all features which would be treated as equivalents thereof by those skilled in the art to which this invention pertains.

What is claimed is:

1. A method for processing a stream from a fermentation process, wherein the stream comprises polysaccharides, the method comprising, contacting the stream with an enzyme to convert at least a portion of the polysaccharides into a fermentable sugar to form a fermentable sugar stream.

2. The method of claim 1, further comprising routing the fermentable sugar stream into the fermentation process.

3. The method of claim 1, wherein the enzyme comprises one or more selected from the group consisting of dextranase, cellulase, galactomannase, hemicellulase, xylanase, fungal amylase, glucoamylase, invertase, bacterial protease, fungal protease, and any combination thereof.

4. The method of claim 1, wherein the enzyme comprises any of the following combinations selected from the group consisting of fungal amylase and glucoamylase; dextranase and invertase; dextranase and cellulase; dextranase, cellulase and glucoamylase; dextranase, cellulase, fungal amylase and glucoamylase; dextranase, cellulase, fungal amylase, glucoamylase, and invertase; hemicellulase, fungal amylase, glucoamylase; cellulase, fungal amylase, glucoamylase; bacterial protease, fungal amylase, glucoamylase; cellulase, bacterial protease, fungal amylase, glucoamylase; hemicellulase, bacterial protease, fungal amylase, glucoamylase; fungal protease, fungal amylase, glucoamylase; cellulase, fungal protease, fungal amylase, glucoamylase; hemicellulase, fungal protease, fungal amylase, glucoamylase; galactomannanase, bacterial protease, fungal amylase, glucoamylase; and galactomannanase, fungal protease, fungal amylase, glucoamylase.

5. The method of claim 1, wherein the fermentation process is a molasses based fermentation process.

6. The method of claim 5, wherein the fermentation process is a rum fermentation process.

7. The method of claim 5, wherein the fermentation process is an ethanol fermentation process.

8. The method of claim 5 wherein the enzyme comprises one or more selected from the group consisting of dextranase, cellulase, galactomannase, hemicellulase, xylanase, fungal amylase, glucoamylase, invertase, bacterial protease, fungal protease, and any combination thereof.

9. The method of claim 8, wherein the stream is subjected to solids reduction and polysaccharide concentration prior to contacting with the enzyme.

10. The method of claim 1, wherein the stream is subjected to solids reduction and polysaccharide concentration prior to contacting with the enzyme.

11. The method of claim 1 wherein the stream is subjected to elevated temperature and acidic conditions to prior to contacting with the enzyme.

12. A fermentation method comprising:
A. Contacting a fermentable sugar and yeast in a fermenter broth to form ethanol and a polysaccharide;
B. Recovering at least a portion of the polysaccharide as a recovered polysaccharide; and
C. Contacting the recovered polysaccharide with an enzyme.

13. The method of claim 12 wherein in Step C at least a portion of the recovered polysaccharide is converted into a formed fermentable sugar, the method further comprising:
D. Adding the formed fermentable sugar to the fermenter broth of step A.

14. The method of claim 12 wherein the enzyme comprises one or more selected from the group consisting of dextranase, cellulase, galactomannase, hemicellulase, xylanase, fungal amylase, glucoamylase, invertase, bacterial protease, fungal protease, and any combination thereof.

15. The method of claim 12 wherein the enzyme comprises any of the following combinations selected from the group consisting of fungal amylase and glucoamylase; dextranase and invertase; dextranase and cellulase; dextranase, cellulase and glucoamylase; dextranase, cellulase, fungal amylase and glucoamylase; dextranase, cellulase, fungal amylase, glucoamylase, and invertase; hemicellulase, fungal amylase, glucoamylase; cellulase, fungal amylase, glucoamylase; bacterial protease, fungal amylase, glucoamylase; cellulase, bacterial protease, fungal amylase, glucoamylase; hemicellulase, bacterial protease, fungal amylase, glucoamylase; fungal protease, fungal amylase, glucoamylase; cellulase, fungal protease, fungal amylase, glucoamylase; hemicellulase, fungal protease, fungal amylase, glucoamylase; galactomannanase, bacterial protease, fungal amylase, glucoamylase; and galactomannanase, fungal protease, fungal amylase, glucoamylase.

16. A product comprising a stream from a fermentation process comprising polysaccharides, and an enzyme.

17. A product of claim 16, wherein the stream is from a molasses based fermentation process.

18. A product of claim 17, wherein the stream is from a rum fermentation process.

19. A product of claim 17, wherein the stream is from an ethanol fermentation process.

20. The product of claim 17, wherein the enzyme comprises one or more selected from the group consisting of dextranase, cellulase, galactomannase, hemicellulase, xylanase, fungal amylase, glucoamylase, invertase, bacterial protease, fungal protease, and any combination thereof.

21. The product of claim 17, wherein the enzyme comprises any of the following combinations selected from the group consisting of fungal amylase and glucoamylase; dextranase and invertase; dextranase and cellulase; dextranase, cellulase and glucoamylase; dextranase, cellulase, fungal amylase and glucoamylase; dextranase, cellulase, fungal amylase, glucoamylase, and invertase; hemicellulase, fungal amylase, glucoamylase; cellulase, fungal amylase, glucoamylase; bacterial protease, fungal amylase, glucoamylase; cellulase, bacterial protease, fungal amylase, glucoamylase; hemicellulase, bacterial protease, fungal amylase, glucoamylase; fungal protease, fungal amylase, glucoamylase; cellulase, fungal protease, fungal amylase, glucoamylase; hemicellulase, fungal protease, fungal amylase, glucoamylase; galactomannanase, bacterial protease, fungal amylase, glucoamylase; and galactomannanase, fungal protease, fungal amylase, glucoamylase.

22. A method for processing a stream from a fermentation process, wherein the stream comprises polysaccharides and organic acids, the method comprising, separating the stream into a concentrated polysaccharide portion having a higher polysaccharide concentration than the stream, and an organic acids portion having a higher acids concentration than the stream.

23. The method of claim 22, wherein the fermentation process is a rum making process.
